(19) United States
(12) Patent Application Publication
(10) Pub. No.: US 2022/0175835 A1
(43) Pub. Date: Jun. 9, 2022

(54) INDUCTION OF INFECTIOUS TOLERANCE BY EX VIVO REPROGRAMMED IMMUNE CELLS

(71) Applicant: CREATIVE MEDICAL TECHNOLOGIES, INC., PHOENIX, AZ (US)

(72) Inventors: Thomas Ichim, San Diego, CA (US); Amit Patel, Salt Lake City, UT (US)

(73) Assignee: CREATIVE MEDICAL TECHNOLOGIES, INC., PHOENIX, AZ (US)

(21) Appl. No.: 17/546,469

(22) Filed: Dec. 9, 2021

Related U.S. Application Data
(60) Provisional application No. 63/123,380, filed on Dec. 9, 2020.

(51) Int. Cl. A61K 35/17; A61K 35/15; C12N 5/0784; A61P 37/00

(52) U.S. Cl. CPC A61K 35/17; A61K 35/15; C12N 5/0639; A61P 37/00; C12N 2501/231; C12N 2501/105; C12N 2501/12; C12N 2501/13; C12N 2501/165

(57) ABSTRACT

Disclosed are means, methods and compositions of matter useful for inhibiting, in an antigen-specific manner, immunity towards an autoantigen or alloantigen. In one embodiment of the invention, regenerative cells are cultured ex vivo together with immune cells from a mammal suffering from an autoimmune condition. Autoantigens or alloantigens are added in the culture of regenerative cells and cells from an autoimmune disease suffering individual in a manner so that said regenerative cells can endow onto said immune cells of said patient suffering from autoimmunity a state of antigen specific infectious tolerance. In one embodiment, said infectious tolerance involves T regulatory cells inducing conversion of dendritic cells to tolerogeneic dendritic cells, and furthermore in other embodiments administration of tolerogenic dendritic cells induces T regulatory cells.

### CROSS-REFERENCE TO RELATED APPLICATIONS

This application claims the benefit of priority to U.S. Provisional Application Ser. No. 63/123,380, filed Dec. 9, 2020, the contents of which are hereby incorporated by reference.

### FIELD OF THE INVENTION

The invention belongs to the field of immunology, more particularly, the invention belongs to the field of transplantation tolerance and tolerance to autoantigen. More particularly, the invention belongs to the field of induction of infectious tolerance, whereby selective immunologic unresponsiveness can be transferred from cell to cell and/or from host to host.

### BACKGROUND OF THE INVENTION

The ability of the immune system to distinguish between self and non-self is paramount to survival. What exactly constitutes “self” and “non-self” is someone plastic in nature. For example, in pregnancy the fetus is clearly “non-self” yet it does not get rejected by the mother. In the few cases that it does, this is an abnormality in what is called the “tolerogenic” cells of the body whose role is to instruct the immune system not to attack certain antigens. One of the key cells involved in tolerance is the dendritic cell. Dendritic cells principally are kind of special antigen-presenting cells performing the function of presenting antigen to a T cell, and present in a type of a spatial dendrite in a lymphatic gland, a spleen, the thymus, under the skin, or the intercellular spaces of various tissues. The dendritic cells play an important role in activating T cells by presenting various antigen samples along with MHC (major histocompatibility complex) Class I or MHC Class II complex to a T cell by absorbing an antigen in cells.

It is known that the dendritic cells are differentiated in a state of different maturities according to a type of environmental signal present around the cells, and thus, exist as immature, semi-mature, or mature dendritic cells. The immature dendritic cells are discovered at an initial maturity state and perform a primary function collecting and removing debris from transcellular fluid. However, the immature dendritic cells express the inflammatory cytokine in the low level, and thus, even when the cells contact with a T cell, the cells cannot activate a T cell. On the other hand, the mature dendritic cells allow a naive T cell to be activated, and thus, have ability capable of inducing an immune reaction. In order for the immature dendritic cells to be differentiated into the mature dendritic cells, the immature dendritic cells should be exposed to many specific signals to activate a toll-like acceptor. For this reason, many co-stimulatory molecules and proinflammatory cytokine like IL-12 are up-regulated and transferred from tissues to a lymphatic gland, and thus, the projections like fingers, which are the morphological feature of the mature dendritic cells, are appeared.

Generation of tolerogenic dendritic cells has been reported in the literature, however scaleable production of these cells is not readily available for therapeutic purposes. Here we describe utilization of ex vivo reprogramming of immune cells in order to create tolerogenic dendritic cells in vitro, but more importantly, in vivo.

### SUMMARY

Preferred embodiments are directed to a method of treating autoimmunity or alloreactivity in a mammal comprising the steps of: a) obtaining immune cells from an individual suffering from autoimmunity or alloimmunity; b) contacting said immune cells with a regenerative cell; c) providing an antigen presenting cell in the mixture in a manner in which said antigen presenting cell is capable of generating a tolerogenic reaction said immune cells from said patient suffering from autoimmunity or alloimmunity.

Preferred methods include embodiments wherein said immune cells are peripheral blood mononuclear cells.

Preferred methods include embodiments wherein said immune cells are T cells.

Preferred methods include embodiments wherein said T cells are CD4 cells.

Preferred methods include embodiments wherein said T cells are CD8 cells.

Preferred methods include embodiments wherein said T cells are NKT cells.

Preferred methods include embodiments wherein said T cells are gamma delta T cells.

Preferred methods include embodiments wherein said T cells are Th1 cells.

Preferred methods include embodiments wherein said Th1 cells have a propensity for producing interferon gamma over interleukin 4 upon stimulation via the CD3 protein.

Preferred methods include embodiments wherein said Th1 cells express STAT4.

Preferred methods include embodiments wherein said Th1 cells express STAT1.

Preferred methods include embodiments wherein said Th1 cells express T-bet.

Preferred methods include embodiments wherein said Th1 cells express CCR1.

Preferred methods include embodiments wherein said Th1 cells express CCR5.

Preferred methods include embodiments wherein said Th1 cells express CXCR3.

Preferred methods include embodiments wherein said Th1 cells express CD119.

Preferred methods include embodiments wherein said Th1 cells express interferon gamma receptor II.

Preferred methods include embodiments wherein said Th1 cells express IL-18 receptor.

Preferred methods include embodiments wherein said Th1 cells express IL-12 receptor.

Preferred methods include embodiments wherein said Th1 cells express IL-27 receptor.

Preferred methods include embodiments wherein said T cells are Th2 cells.

Preferred methods include embodiments wherein said Th2 cells have a proclivity to produce more interleukin-4 than interferon gamma upon stimulation via CD3.

Preferred methods include embodiments wherein said Th2 cells express GATA-3.

Preferred methods include embodiments wherein said Th2 cells express IRF-4.

Preferred methods include embodiments wherein said Th2 cells express STAT5.

Preferred methods include embodiments wherein said Th2 cells express STAT6.

Preferred methods include embodiments wherein said Th2 cells express CCR3.

Preferred methods include embodiments wherein said Th2 cells express CCR4.

Preferred methods include embodiments wherein said Th2 cells express CCR8.

Preferred methods include embodiments wherein said Th2 cells express CXCR4.

Preferred methods include embodiments wherein said Th2 cells express interleukin-4 receptor.

Preferred methods include embodiments wherein said Th2 cells express interleukin-33 receptor.

Preferred methods include embodiments wherein said T cells are Th9 cells.

Preferred methods include embodiments wherein said Th9 cell produces interleukin-9.

Preferred methods include embodiments wherein said Th9 cell expresses IRF4.

Preferred methods include embodiments wherein said Th9 cell expresses PU.1.

Preferred methods include embodiments wherein said Th9 cell secretes CCL17.

Preferred methods include embodiments wherein said Th9 cell secretes CCL22.

Preferred methods include embodiments wherein said Th9 cell secretes IL-10.

Preferred methods include embodiments wherein said Th9 cell expresses TGF-beta receptor II.

Preferred methods include embodiments wherein said T cell is a follicular helper T cell.

Preferred methods include embodiments wherein said follicular helper T cell expresses bcl-6.

Preferred methods include embodiments wherein said follicular helper T cell expresses c-maf.

Preferred methods include embodiments wherein said follicular helper T cell expresses stat-3.

Preferred methods include embodiments wherein said follicular helper T cell secretes CXCL-13.

Preferred methods include embodiments wherein said follicular helper T cell secretes interferon gamma.

Preferred methods include embodiments wherein said follicular helper T cell secretes interleukin-4.

Preferred methods include embodiments wherein said follicular helper T cell secretes IL-10.

Preferred methods include embodiments wherein said follicular helper T cell secretes IL-17A.

Preferred methods include embodiments wherein said follicular helper T cell secretes IL-17F.

Preferred methods include embodiments wherein said follicular helper T cell secretes IL-21.

Preferred methods include embodiments wherein said follicular helper T cell expresses BTLA-4.

Preferred methods include embodiments wherein said follicular helper T cell secretes CD40 ligand.

Preferred methods include embodiments wherein said follicular helper T cell expresses CD57.

Preferred methods include embodiments wherein said follicular helper T cell expresses CD84.

Preferred methods include embodiments wherein said follicular helper T cell expresses CXCR-4.

Preferred methods include embodiments wherein said follicular helper T cell expresses CXCR-5.

Preferred methods include embodiments wherein said follicular helper T cell expresses ICOS.

Preferred methods include embodiments wherein said follicular helper T cell expresses IL-6 receptor.

Preferred methods include embodiments wherein said follicular helper T cell expresses IL-21 receptor.

Preferred methods include embodiments wherein said follicular helper T cell expresses CD10.

Preferred methods include embodiments wherein said follicular helper T cell expresses OX40.

Preferred methods include embodiments wherein said follicular helper T cell expresses PD-1.

Preferred methods include embodiments wherein said follicular helper T cell expresses CD150.

Preferred methods include embodiments wherein said T cell is a Th17 cell.

Preferred methods include embodiments wherein said Th17 cell secretes interleukin-17A.

Preferred methods include embodiments wherein said Th17 cell secretes interleukin-17F.

Preferred methods include embodiments wherein said Th17 cell secretes IL-21.

Preferred methods include embodiments wherein said Th17 cell secretes IL-26.

Preferred methods include embodiments wherein said Th17 cell secretes CCL20.

Preferred methods include embodiments wherein said Th17 cell expresses BATF.

Preferred methods include embodiments wherein said Th17 cell expresses IRF4.

Preferred methods include embodiments wherein said Th17 cell expresses ROR alpha.

Preferred methods include embodiments wherein said Th17 cell expresses ROR gamma.

Preferred methods include embodiments wherein said Th17 cell expresses STAT5.

Preferred methods include embodiments wherein said Th17 cell expresses CCR4.

Preferred methods include embodiments wherein said Th17 cell expresses CCR6.

Preferred methods include embodiments wherein said Th17 cell expresses IL-1 receptor.

Preferred methods include embodiments wherein said Th17 cell expresses IL-6 receptor alpha.

Preferred methods include embodiments wherein said Th17 cell expresses IL-21 receptor.

Preferred methods include embodiments wherein said Th17 cell expresses IL-23 receptor.

Preferred methods include embodiments wherein said T cell is a Th22 cell.

Preferred methods include embodiments wherein said Th22 cell secretes IL-10.

Preferred methods include embodiments wherein said Th22 cell secretes IL-13.

Preferred methods include embodiments wherein said Th22 cell secretes FGF-1.

Preferred methods include embodiments wherein said Th22 cell secretes FGF-2.

Preferred methods include embodiments wherein said Th22 cell secretes FGF-5.

Preferred methods include embodiments wherein said Th22 cell secretes IL-21.

Preferred methods include embodiments wherein said Th22 cell secretes IL-22.

Preferred methods include embodiments wherein said Th22 cell expresses AHR.

Preferred methods include embodiments wherein said Th22 cell expresses batf.

Preferred methods include embodiments wherein said Th22 cell expresses STAT-3

Preferred methods include embodiments wherein said Th22 cell expresses CCR4.

Preferred methods include embodiments wherein said Th22 cell expresses CCR6.

Preferred methods include embodiments wherein said Th22 cell expresses CCR10.

Preferred methods include embodiments wherein said Th22 cell expresses IL-6 receptor.

Preferred methods include embodiments wherein said Th22 cell expresses TGF-beta receptor II.

Preferred methods include embodiments wherein said Th22 cell expresses TNF receptor 1.

Preferred methods include embodiments wherein said T cell is a T regulatory cell.

Preferred methods include embodiments wherein said T regulatory cell expresses foxp-3.

Preferred methods include embodiments wherein said T regulatory cell expresses Helios.

Preferred methods include embodiments wherein said T regulatory cell expresses STAT5.

Preferred methods include embodiments wherein said T regulatory cell expresses CD5.

Preferred methods include embodiments wherein said T regulatory cell expresses CD25.

Preferred methods include embodiments wherein said T regulatory cell expresses CD39.

Preferred methods include embodiments wherein said T regulatory cell expresses CD105.

Preferred methods include embodiments wherein said T regulatory cell expresses IL-7 receptor.

Preferred methods include embodiments wherein said T regulatory cell expresses CTLA-4.

Preferred methods include embodiments wherein said T regulatory cell expresses folate receptor.

Preferred methods include embodiments wherein said T regulatory cell expresses CD223

Preferred methods include embodiments wherein said T regulatory cell expresses LAP.

Preferred methods include embodiments wherein said T regulatory cell expresses GARP.

Preferred methods include embodiments wherein said T regulatory cell expresses Neuropilin.

Preferred methods include embodiments wherein said T regulatory cell expresses CD134.

Preferred methods include embodiments wherein said T regulatory cell expresses CD62 ligand.

Preferred methods include embodiments wherein said T regulatory cell secretes IL-10.

Preferred methods include embodiments wherein said T regulatory cell secretes TGF-alpha.

Preferred methods include embodiments wherein said T regulatory cell secretes TGF-beta.

Preferred methods include embodiments wherein said T regulatory cell secretes soluble TNF receptor p55.

Preferred methods include embodiments wherein said T regulatory cell secretes soluble TNF receptor p75.

Preferred methods include embodiments wherein said T regulatory cell secretes IL-2.

Preferred methods include embodiments wherein said T regulatory cell secretes soluble HLA-G.

Preferred methods include embodiments wherein said T regulatory cell secretes soluble Fas ligand.

Preferred methods include embodiments wherein said T regulatory cell secretes IL-35.

Preferred methods include embodiments wherein said T regulatory cell secretes VEGF.

Preferred methods include embodiments wherein said T regulatory cell secretes HGF.

Preferred methods include embodiments wherein said T regulatory cell secretes FGF1.

Preferred methods include embodiments wherein said T regulatory cell secretes FGF2.

Preferred methods include embodiments wherein said T regulatory cell secretes FGF5.

Preferred methods include embodiments wherein said T regulatory cell secretes Galectin 1.

Preferred methods include embodiments wherein said T regulatory cell secretes Galectin 9.

Preferred methods include embodiments wherein said T regulatory cell secretes IL-20.

Preferred methods include embodiments wherein said T regulatory cell expresses perforin.

Preferred methods include embodiments wherein said T regulatory cell expresses granzyme.

Preferred methods include embodiments wherein said T regulatory cell inhibits activation of a conventional T cell.

Preferred methods include embodiments wherein said conventional T cell does not express CD25.

Preferred methods include embodiments wherein said conventional T cell is activated by ligation of CD3.

Preferred methods include embodiments wherein said T conventional T cell is activated by ligation of CD3 and CD28.

Preferred methods include embodiments wherein said activation of said conventional T cell is proliferation.

Preferred methods include embodiments wherein said activation of said conventional T cell is cytokine secretion.

Preferred methods include embodiments wherein said cytokine is IL-2.

Preferred methods include embodiments wherein said cytokine is IL-4.

Preferred methods include embodiments wherein said cytokine is IL-6.

Preferred methods include embodiments wherein said cytokine is IL-10.

Preferred methods include embodiments wherein said cytokine is IL-12.

Preferred methods include embodiments wherein said cytokine is IL-7.

Preferred methods include embodiments wherein said cytokine is IL-13.

Preferred methods include embodiments wherein said cytokine is IL-15.

Preferred methods include embodiments wherein said cytokine is IL-18.

Preferred methods include embodiments wherein said immune cells are peripheral blood mononuclear cells.

Preferred methods include embodiments wherein said peripheral blood mononuclear cells are treated with an immune modulatory agent prior to contacting with said regenerative cells.

Preferred methods include embodiments wherein said immune modulatory agent is ultraviolet light.

Preferred methods include embodiments wherein said immune modulatory agent is HGF.

Preferred methods include embodiments wherein said immune modulatory agent is oxytocin.

Preferred methods include embodiments wherein said immune modulatory agent is NGF.

Preferred methods include embodiments wherein said immune modulatory agent is FGF-1.

Preferred methods include embodiments wherein said immune modulatory agent is FGF-2.

Preferred methods include embodiments wherein said immune modulatory agent is a toll like receptor activator.

Preferred methods include embodiments wherein oxytocin is administered to peripheral blood mononuclear cells in vitro for a period of 1 minute to 4 weeks.

Preferred methods include embodiments wherein oxytocin is administered to peripheral blood mononuclear cells in vitro for a period of 2 hours to 1 week.

Preferred methods include embodiments wherein oxytocin is administered to said fibroblasts in vitro for a period of 24 hours to 72 hours.

Preferred methods include embodiments wherein said oxytocin is administered at a concentration of 10 nM-10 uM.

Preferred methods include embodiments wherein said oxytocin is administered at a concentration of 100 nM-1 uM.

Preferred methods include embodiments wherein said immune modulatory agent is morphine.

Preferred methods include embodiments wherein said immune modulatory agent is curcumin.

Preferred methods include embodiments wherein said immune modulatory agent is TGF-beta.

Preferred methods include embodiments wherein said immune modulatory agent is galectin-1.

Preferred methods include embodiments wherein said immune modulatory agent is galectin-3.

Preferred methods include embodiments wherein said immune modulatory agent is galectin-9.

Preferred methods include embodiments wherein said immune modulatory agent is IL-1.

Preferred methods include embodiments wherein said immune modulatory agent is IL-2.

Preferred methods include embodiments wherein said immune modulatory agent is IL-4.

Preferred methods include embodiments wherein said immune modulatory agent is IL-7.

Preferred methods include embodiments wherein said immune modulatory agent is IL-10.

Preferred methods include embodiments wherein said immune modulatory agent is IL-13.

Preferred methods include embodiments wherein said immune modulatory agent is IL-15.

Preferred methods include embodiments wherein said immune modulatory agent is IL-12.

Preferred methods include embodiments wherein said immune modulatory agent is IL-18.

Preferred methods include embodiments wherein said immune modulatory agent is IL-20.

Preferred methods include embodiments wherein said immune modulatory agent is IL-22.

Preferred methods include embodiments wherein said immune modulatory agent is IL-35.

Preferred methods include embodiments wherein said immune modulatory agent is conditioned media from mesenchymal stem cells.

Preferred methods include embodiments wherein said mesenchymal stem cells are plastic adherent.

Preferred methods include embodiments wherein said mesenchymal stem cells are derived from adipose tissue.

Preferred methods include embodiments wherein said mesenchymal stem cells are derived from bone marrow.

Preferred methods include embodiments wherein said mesenchymal stem cells are derived from placenta.

Preferred methods include embodiments wherein said mesenchymal stem cells are derived from Wharton's Jelly.

Preferred methods include embodiments wherein said mesenchymal stem cells are derived from endometrium.

Preferred methods include embodiments wherein said mesenchymal stem cells are derived from menstrual blood.

Preferred methods include embodiments wherein said mesenchymal stem cells are derived from fallopian tube.

Preferred methods include embodiments wherein said mesenchymal stem cells are derived from deciduous tooth.

### BRIEF DESCRIPTION OF THE DRAWINGS

FIG. 1 is a graph showing resolution of rheumatoid arthritis by administration of amniotic stem cell conditioned splenocytes.

FIG. 2 is a bar graph showing administration of amniotic stem cell conditioned splenocytes increases tolerogenic dendritic cells in vivo.

### DESCRIPTION OF THE INVENTION

The invention, in some embodiments, teaches the application of Immunological tolerance to the condition of alloantigen reactivity and autoimmunity. It is known that a cardinal feature of the immune system, is allowing for recognition and elimination of pathological threats, while selectively ignoring antigens that belong to the body. Traditionally, autoimmune conditions or conditions associated with cytokine storm, or allograft rejection are treated with non-specific inhibitors of inflammation such as steroids, as well as immune suppressive agents such as cyclosporine, 5-azathrioprine, and methotrexate. These approaches globally suppress immune functions and have numerous undesirable side effects. Unfortunately, given the substantial decrease in quality of life observed in patients with autoimmunity, the potential of alleviation of autoimmune symptoms outweighs the side effects such as opportunistic infections and increased predisposition to neoplasia.

The invention provides novel stem cell types, methods of manufacture, and therapeutic uses. Provided are means of deriving stem cells possessing regenerative, immune modulatory, anti-inflammatory, and angiogenic/neurogenic activity from umbilical cord tissue such as Wharton's Jelly. In some embodiments manipulation of stem cell “potency” is disclosed through hypoxic manipulation, growth on non-xenogeneic conditions, as well as addition of epigenetic modulators.

The cells of the invention are cultured under hypoxia, in one embodiment, cultured in order to induce and/or augment expression of chemokine receptors. One such receptor is CXCR-4. The population of cells, including population of umbilical cord mesenchymal cells, may be enriched for CXCR-4, such as (or such as about) 50%, 55%, 60%, 65%, 70%, 75%, 80%, 85%, 90%, 95%, 96%, 97%, 98%, 99%, or more of the population expressing CXCR-4, CD31, CD34, or any combination thereof. In addition or alternatively, <1%, <2%, <3%, <4%, <5%, <6%, <7%, <8%, <9%, or <10% of the population of cells may express CD14 and/or CD45. The umbilical cord cells of the invention may further possess markers selected from the group consisting of STRO-1, CD105, CD54, CD56, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, and a combination thereof. In some embodiments said placental cells of the invention are admixed with endothelial cells. Said endothelial cells may express one or more markers selected from the group consisting of: a) extracellular vimentin; b) CD133; c) c-kit; d) VEGF receptor; e) activated protein C receptor; and f) a combination thereof. In some embodiments, the population of endothelial cells comprises endothelial progenitor cells.

The population of cells may be allogeneic, autologous, or xenogenic to an individual, including an individual being administered the population of cells. In some embodiments, the population of cells are matched by mixed lymphocyte reaction matching.

In some embodiments, the population of cells is derived from tissue selected from the group consisting of the placental body, placenta, umbilical cord tissue, peripheral blood, hair follicle, cord blood, Wharton's Jelly, menstrual blood, endometrium, skin, omentum, amniotic fluid, and a combination thereof. In some embodiments, the population of cells, the population of umbilical mesenchymal stem cells, or the population of endothelial cells comprises human umbilical cord derived adherent cells. The human umbilical cord derived adherent cells may express a cytokines selected from the group consisting of) FGF-1; b) FGF-2; c) HGF; d) interleukin-1 receptor antagonist; and e) a combination thereof. In some embodiments, the population of cells, the population of umbilical cord cells express arginase, indoleamine 2,3 deoxygenase, interleukin-10, and/or interleukin 35. In some embodiments, the population of cells, the population of umbilical cord cells, or the population of endothelial cells express hTERT and Oct-4 but does not express a STRO-1 marker.

In some embodiments, the population of cells, the population of umbilical cord cells has an ability to undergo cell division in less than 36 hours in a growth medium. In some embodiments, the population of cells, the population of umbilical cord cells has an ability to proliferate at a rate of 0.9-1.2 doublings per 36 hours in growth media. In some embodiments, the population of cells, the population of umbilical cord cells has an ability to proliferate at a rate of 0.9, 1.0, 1.1, or 1.2 doublings per 36 hours in growth media. The population of cells, population of umbilical cord cells may produce exosomes capable of inducing more than 50% proliferation when the exosomes are cultured with human umbilical cord endothelial cells. The induction of proliferation may occur when the exosomes are cultured with the human umbilical cord endothelial cells at a concentration of 10, 20, 30, 40, 50, 60, 70, 80, 90, 100, 110, 120, 130, 140, 150, 160, 170, 180, 190, 200, or more exosomes per cell.

In some embodiments, a population of cells, including a population of umbilical cells alone, are administered to an individual, including an individual having and acute or chronic pathology, wherein the population of cells may be administered via any suitable route, including as non-limiting examples, intramuscularly and/or intravenously.

In some embodiments, a population of umbilical cord cells is optionally obtained, the population is then optionally contacted via culturing with a population of progenitor for T regulatory cells, wherein the culturing conditions allow for the generation of T regulatory cells, then the generated T regulatory cells are administered to an individual.

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

Chemical Modification: As used herein, “chemical modification” refers to the process wherein a chemical or biochemical is used to induce genomic changes in the donor cell, or nucleus thereof, that allow the donor cell, or nucleus thereof, to be responsive during maturation and receptive to the host cell cytoplasm.

Committed: As used herein, “committed” refers to cells which are considered to be permanently committed to a specific function. Committed cells are also referred to as “terminally differentiated cells.”

Cytoplast Extract Modification: As used herein, “cytoplast extract modification” refers to the process wherein a cellular extract consisting of the cytoplasmic contents of a cell are used to induce genomic changes in the donor cell, or nucleus thereof, that allow the donor cell, or nucleus thereof, to be responsive during maturation and receptive to the host cell cytoplasm.

Dedifferentiation: As used herein, “dedifferentiation” refers to loss of specialization in form or function. In cells, dedifferentiation leads to an a less committed cell.

Differentiation: As used herein, “differentiation” refers to the adaptation of cells for a particular form or function. In cells, differentiation leads to a more committed cell.

Donor Cell: As used herein, “donor cell” refers to any diploid (2N) cell derived from a pre-embryonic, embryonic, fetal, or post-natal multi-cellular organism or a primordial sex cell which contributes its nuclear genetic material to the hybrid stem cell. The donor cell is not limited to those cells that are terminally differentiated or cells in the process of differentiation. For the purposes of this invention, donor cell refers to both the entire cell or the nucleus alone.

Donor Cell Preparation: As used herein, “donor cell preparation” refers to the process wherein the donor cell, or nucleus thereof, is prepared to undergo maturation or prepared to be receptive to a host cell cytoplasm and/or responsive within a post-natal environment.

Germ Cell: As used herein, “germ cell” refers to a reproductive cell such as a spermatocyte or an oocyte, or a cell that will develop into a reproductive cell.

Host Cell: As used herein, “host cell” refers to any multipotent stem cell derived from a pre-embryonic, embryonic, fetal, or post-natal multicellular organism that contributes the cytoplasm to a hybrid stem cell.

Host Cell Preparation: As used herein, “host cell preparation” refers to the process wherein the host cell is enucleated.

Hybrid Stem Cell: As used herein, “hybrid stem cell” refers to any cell that is multipotent and is derived from an enucleated host cell and a donor cell, or nucleus thereof, of a multicellular organism. Hybrid stem cells are further disclosed in co-pending U.S. patent application Ser. No. 10/864,788.

Karyoplast Extract Modification: As used herein, “karyoplast extract modification” refers to the process wherein a cellular extract consisting of the nuclear contents of a cell, lacking the DNA, are used to induce genomic changes in the donor cell, or nucleus thereof, that allow the donor cell, or nucleus thereof, to be responsive during maturation or receptive to the host cell cytoplasm.

Maturation: As used herein, “maturation” refers to a process of coordinated steps either forward or backward in the differentiation pathway and can refer to both differentiation or de-differentiation. As used herein, maturation is synonymous with the terms develop or development when applied to the process described herein.

Modified Germ Cell: As used herein, “modified germ cell” refers to a cell comprised of a host enucleated ovum and a donor nucleus from a spermatogonia, oogonia or a primordial sex cell. The host enucleated ovum and donor nucleus can be from the same or different species. A modified germ cell can also be called a “hybrid germ cell.”

Multipotent: As used herein, “multipotent” refers to cells that can give rise to several other cell types, but those cell types are limited in number. An example of a multipotent cells is hematopoietic cells—blood stem cells that can develop into several types of blood cells but cannot develop into brain cells.

Multipotent Adult Progenitor Cells: As used herein, “multipotent adult progenitor cells” refers to multipotent cells isolated from the bone marrow which have the potential to differentiate into mesenchymal, endothelial and endodermal lineage cells.

Pre-embryo: As used herein, “pre-embryo” refers to a fertilized egg in the early stage of development prior to cell division. During the pre-embryonic stage the initial stages of cleavage are occurring.

Pre-embryonic Stem Cell: See “Embryonic Stem Cell” above.

Post-natal Stem Cell: As used herein, “post-natal stem cell” refers to any cell that is multipotent and derived from a multi-cellular organism after birth.

Pluripotent: As used herein, “pluripotent” refers to cells that can give rise to any cell type except the cells of the placenta or other supporting cells of the uterus.

Primordial Sex Cell: As used herein, “primordial sex cell” refers to any diploid cell that is derived from the male or female mature or developing gonad, is able to generate cells that propagate a species and contains a diploid genomic state. Primordial sex cells can be quiescent or actively dividing. These cells include male gonocytes, female gonocytes, spermatogonial stem cells, ovarian stem cells, oogonia, type-A spermatogonia, Type-B spermatogonia. Also known as germ-line stem cells.

Primordial Germ Cell: As used herein, “primordial germ cell” refers to cells present in early embryogenesis that are destined to become germ cells.

Reprogamming: As used herein “reprogramming” refers to the resetting of the genetic program of a cell such that the cell exhibits pluripotency and has the potential to produce a fully developed organism.

Responsive: As used herein, “responsive” refers to the condition of a cell, or group of cells, wherein they are susceptible to and can function accordingly within a cellular environment. Responsive cells are capable of responding to and functioning in a particular cellular environment, tissue, organ and/or organ system.

Somatic Stem Cells: As used herein, “somatic stem cells” refers to diploid multipotent or pluripotent stem cells. Somatic stem cells are not totipotent stem cells. Stem cells are primitive cells that give rise to other types of cells. Also called progenitor cells, there are several kinds of stem cells. Totipotent cells are considered the “master” cells of the body because they contain all the genetic information needed to create all the cells of the body plus the placenta, which nourishes the human embryo. Human cells have this totipotent capacity only during the first few divisions of a fertilized egg. After three to four divisions of totipotent cells, there follows a series of stages in which the cells become increasingly specialized. The next stage of division results in pluripotent cells, which are highly versatile and can give rise to any cell type except the cells of the placenta or other supporting tissues of the uterus. At the next stage, cells become multipotent, meaning they can give rise to several other cell types, but those types are limited in number. An example of multipotent cells is hematopoietic cells—blood cells that can develop into several types of blood cells, but cannot develop into brain cells. At the end of the long chain of cell divisions that make up the embryo are “terminally differentiated” cells—cells that are considered to be permanently committed to a specific function.

Therapeutic Cloning: As used herein, “therapeutic cloning” refers to the cloning of cells using nuclear transfer methods including replacing the nucleus of an ovum with the nucleus of another cell and stem cells derived from the inner cell mass.

Therapeutic Reprogramming: As used herein, “therapeutic reprogramming” refers to the process of maturation wherein a stem cell is exposed to stimulatory factors according to the teachings of the present invention to yield either pluripotent, multipotent or tissue-specific committed cells. Therapeutically reprogrammed cells are useful for implantation into a host to replace or repair diseased, damaged, defective or genetically impaired tissue. The therapeutically reprogrammed cells of the present invention do not possess non-human sialic acid residues.

Totipotent: As used herein, “totipotent” refers to cells that contain all the genetic information needed to create all the cells of the body plus the placenta. Human cells have the capacity to be totipotent only during the first few divisions of a fertilized egg.

Whole Cell Extract Modification: As used herein, “whole cell extract modification” refers to the process wherein a cellular extract consisting of the cytoplasmic and nuclear contents of a cell are used to induce genomic changes in the donor cell, or nucleus thereof, that allow the donor cell, or nucleus thereof, to be responsive during maturation and receptive to the host cell cytoplasm

In one embodiment the invention teaches phenotypically defined MSC which can be isolated from the Wharton's jelly of umbilical cord segments and defined morphologically and by cell surface markers. By dissecting out the veins and arteries of cord segments and exposing the Wharton's jelly, the cells of invention, of one embodiment of the invention, may be obtained. An approximately 1-5 cm cord segment is placed in collagenase solution (1 mg/ml, Sigma) for approximately 18 hrs at room temperature. After incubation, the remaining tissue is removed and the cell suspension is diluted with PBS into two 50 ml tubes and centrifuged. Cells are then washed in PBS and counted using hematocytometer. 5-20.times.10.sup.6 cells were then plated in a 6 cm tissue culture plate in low-glucose DMEM (Gibco) with 10% FBS (Hyclone), 2 mM L-Glutamine (Gibco), 100 U/ml penicillin/100 ug/ml streptomycin/0.025 ug/ml amphotericin B (Gibco). At this step of the purification process, cells are exposed to hypoxia. The amount of hypoxia needed is the sufficient amount to induce activagion of HIF-1 alpha. In one embodiment cells are cultured for 24 hours at 2% oxygen. After 48 hrs cells are washed with PBS and given fresh media. Cells were given new media twice weekly. After 7 days, cells are approximately 70-80% confluent and are passed using HyQTase (Hyclone) into a 10 cm plate. Cells are then regularly passed 1:2 every 7 days or upon reaching 80% confluence.

In another embodiment of the invention, biologically useful stem cells are disclosed, of the mesenchymal or related lineages, which are therapeutically reprogrammed cells having minimal oxidative damage and telomere lengths that compare favorably with the telomere lengths of undamaged, pre-natal or embryonic stem cells (that is, the therapeutically reprogrammed cells of the present invention possess near prime physiological state genomes). Moreover the therapeutically reprogrammed cells of the present invention are immunologically privileged and therefore suitable for therapeutic applications. Additional methods of the present invention provide for the generation of hybrid stem cells. Furthermore, the present invention includes related methods for maturing stem cells made in accordance with the teachings of the present invention into specific host tissues. For use in the current invention, the practitioner is thought that ontogeny of mammalian development provides a central role for stem cells. Early in embryogenesis, cells from the proximal epiblast destined to become germ cells (primordial germ cells) migrate along the genital ridge. These cells express high levels of alkaline phosphatase as well as expressing the transcription factor Oct4. Upon migration and colonization of the genital ridge, the primordial germ cells undergo differentiation into male or female germ cell precursors (primordial sex cells). For the purpose of this invention disclosure, only male primordial sex cells (PSC) will be discussed, but the qualities and properties of male and female primordial sex cells are equivalent and no limitations are implied. During male primordial sex cell development, the primordial stem cells become closely associated with precursor sertoli cells leading to the beginning of the formation of the seminiferous cords. When the primordial germ cells are enclosed in the seminiferous cords, they differentiate into gonocytes that are mitotically quiescent. These gonocytes divide for a few days followed by arrest at G0/G1 phase of the cell cycle. In mice and rats these gonocytes resume division within a few days after birth to generate spermatogonial stem cells and eventually undergo differentiation and meiosis related to spermatogenesis. It is known that embryonic stem cells are cells derived from the inner cell mass of the pre-implantation blastocyst-stage embryo and have the greatest differentiation potential, being capable of giving rise to cells found in all three germ layers of the embryo proper. From a practical standpoint, embryonic stem cells are an artifact of cell culture since, in their natural epiblast environment, they only exist transiently during embryogenesis. Manipulation of embryonic stem cells in vitro has lead to the generation and differentiation of a wide range of cell types, including cardiomyocytes, hematopoietic cells, endothelial cells, nerves, skeletal muscle, chondrocytes, adipocytes, liver and pancreatic islets. Growing embryonic stem cells in co-culture with mature cells can influence and initiate the differentiation of the embryonic stem cells to a particular lineage. Maturation is a process of coordinated steps either forward or backward in the differentiation pathway and can refer to both differentiation and/or dedifferentiation. In one example of the maturation process, a cell, or group of cells, interacts with its cellular environment during embryogenesis and organogenesis. As maturation progresses, cells begin to form niches and these niches, or microenvironments, house stem cells that direct and regulate organogenesis. At the time of birth, maturation has progressed such that cells and appropriate cellular niches are present for the organism to function and survive post-natally. Developmental processes are highly conserved amongst the different species allowing maturation or differentiation systems from one mammalian species to be extended to other mammalian species in the laboratory. During the lifetime of an organism, the cellular composition of the organs and organs systems are exposed to a wide range of intrinsic and extrinsic factors that induce cellular or genomic damage. Ultraviolet light not only has an effect on normal skin cells but also on the skin stem cell population. Chemotherapeutic drugs used to treat cancer have a devastating effect on hematopoietic stem cells. Reactive oxygen species, which are the byproducts of cellular metabolism, are intrinsic factors that compromises the genomic integrity of the cell. In all organs or organ systems, cells are continuously being replaced from stem cell populations. However, as an organism ages, cellular damage accumulates in these stem cell populations. If the damage is inheritable, such as genomic mutations, then all progeny will be effected and thus compromised. A single stem cell clone can contribute to generations of lineages such as lymphoid and myeloid cells for more than a year and therefore have the potential to spread mutations if the stem cell is damaged. The body responds to a compromised stem cell by inducing apoptosis thereby removing it from the pool and preventing potentially dysfunctional or tumorigenic properties. Apoptosis removes compromised cells from the population, but it also decreases the number of stem cells that are available for the future. Therefore, as an organism ages, the number of stem cells decrease. In addition to the loss of the stem cell pool, there is evidence that aging decreases the efficiency of the homing mechanism of stem cells. Telomeres are the physical ends of chromosomes that contain highly conserved, tandemly repeated DNA sequences. Telomeres are involved in the replication and stability of linear DNA molecules and serve as counting mechanism in cells; with each round of cell division the length of the telomeres shortens and at a pre-determined threshold, a signal is activated to initiate cellular senescence. Stem cells and somatic cells produce telomerase, which inhibits shortening of telomeres, but their telomeres still progressively shorten during aging and cellular stress. In one teaching, or embodiment, of the invention, therapeutically reprogrammed cells, in some embodiments mesenchymal stem cells, are provided. Therapeutic reprogramming refers to a maturation process wherein a stem cell is exposed to stimulatory factors according the teachings of the present invention to yield enhanced therapeutic activity. In some embodiments, enhancement of therapeutic activity may be increase proliferation, in other embodiments, it may be enhanced chemotaxis. Other therapeutic characteristics include ability to under resistance to apoptosis, ability to overcome senescence, ability to differentiate into a variety of different cell types effectively, and ability to secrete therapeutic growth factors which enhance viability/activity, of endogenous stem cells. In order to induce therapeutic reprogramming of cells, in some cases, as disclosed herein, of wharton's jelly originating cells, the invention teaches the utilization of stimulatory factors, including without limitation, chemicals, biochemicals and cellular extracts to change the epigenetic programming of cells. These stimulatory factors induce, among other results, genomic methylation changes in the donor DNA. Embodiments of the present invention include methods for preparing cellular extracts from whole cells, cytoplasts, and karyplasts, although other types of cellular extracts are contemplated as being within the scope of the present invention. In a non-limiting example, the cellular extracts of the present invention are prepared from stem cells, specifically embryonic stem cells. Donor cells are incubated with the chemicals, biochemicals or cellular extracts for defined periods of time, in a non-limiting example for approximately one hour to approximately two hours, and those reprogrammed cells that express embryonic stem cell markers, such as Oct4, after a culture period are then ready for transplantation, cryopreservation or further maturation. In another embodiment of the present invention, hybrid stem cells are provided which can be used for cellular regenerative/reparative therapy. The hybrid stem cells of the present invention are pluripotent and customized for the intended recipient so that they are immunologically compatible with the recipient. Hybrid stem cells are a fusion product between a donor cell, or nucleus thereof, and a host cell. Typically the fusion occurs between a donor nucleus and an enucleated host cell. The donor cell can be any diploid cell, including but not limited to, cells from pre-embryos, embryos, fetuses and post-natal organisms. More specifically, the donor cell can be a primordial sex cell, including but not limited to, oogonium or differentiated or undifferentiated spermatogonium, or an embryonic stem cell. Other non-limiting examples of donor cells are therapeutically reprogrammed cells, embryonic stem cells, fetal stem cells and multipotent adult progenitor cells. Preferably the donor cell has the phenotype of the intended recipient. The host cell can be isolated from tissues including, but not limited to, pre-embryos, embryos, fetuses and post-natal organisms and more specifically can include, but is not limited to, embryonic stem cells, fetal stem cells, multipotent adult progenitor cells and adipose-derived stem cells. In a non-limiting example, cultured cell lines can be used as donor cells. The donor and host cells can be from the same individual or different individuals. In one embodiment of the present invention, lymphocytes are used as donor cells and a two-step method is used to purify the donor cells. After the tissues was disassociated, an adhesion step was performed to remove any possible contaminating adherent cells followed by a density gradient purification step. The majority of lymphocytes are quiescent (in GO phase) and therefore can have a methylation status than conveys greater plasticity for reprogramming. Multipotent or pluripotent stem cells or cell lines useful as donor cells in embodiments of the present invention are functionally defined as stem cells by their ability to undergo differentiation into a variety of cell types including, but not limited to, adipogenic, neurogenic, osteogenic, chondrogenic and cardiogenic cell.

In some embodiments, host cell enucleation for the generation of hybrid stem cells according to the teachings of the present invention can be conducted using a variety of means. In a non-limiting example, ADSCs were plated onto fibronectin coated tissue culture slides and treated with cells with either cytochalasin D or cytochalasin B. After treatment, the cells can be trypsinized, re-plated and are viable for about 72 hours post enucleation. Host cells and donor nuclei can be fused using one of a number of fusion methods known to those of skill in the art, including but not limited to electrofusion, microinjection, chemical fusion or virus-based fusion, and all methods of cellular fusion are envisioned as being within the scope of the present invention. The hybrid stem cells made according to the teachings of the present invention possess surface antigens and receptors from the enucleated host cell but has a nucleus from a developmentally younger cell. Consequently, the hybrid stem cells of the present invention will be receptive to cytokines, chemokines and other cell signaling agents, yet possess a nucleus free from age-related DNA damage. The therapeutically reprogrammed cells and hybrid stem cells made in accordance with the teachings of the present invention are useful in a wide range of therapeutic applications for cellular regenerative/reparative therapy. For example, and not intended as a limitation, the therapeutically reprogrammed cells and hybrid stem cells of the present invention can be used to replenish stem cells in animals whose natural stem cells have been depleted due to age or ablation therapy such as cancer radiotherapy and chemotherapy. In another non-limiting example, the therapeutically reprogrammed cells and hybrid stem cells of the present invention are useful in organ regeneration and tissue repair. In one embodiment of the present invention, therapeutically reprogrammed cells and hybrid stem cells can be used to reinvigorate damaged muscle tissue including dystrophic muscles and muscles damaged by ischemic events such as myocardial infarcts. In another embodiment of the present invention, the therapeutically reprogrammed cells and hybrid stem cells disclosed herein can be used to ameliorate scarring in animals, including humans, following a traumatic injury or surgery. In this embodiment, the therapeutically reprogrammed cells and hybrid stem cells of the present invention are administered systemically, such as intravenously, and migrate to the site of the freshly traumatized tissue recruited by circulating cytokines secreted by the damaged cells. In another embodiment of the present invention, the therapeutically reprogrammed cells and hybrid stem cells can be administered locally to a treatment site in need or repair or regeneration.

In one embodiment, umbilical cord samples were obtained following the delivery of normal term babies with Institutional Review Board approval. A portion of the umbilical cord was then cut into approximately 3 cm long segments. The segments were then placed immediately into 25 ml of phosphate buffered saline without calcium and magnesium (PBS) and 1.times. antibiotics (100 U/ml penicillin, 100 ug/ml streptomycin, 0.025 ug/ml amphotericin B). The tubes were then brought to the lab for dissection within 6 hours. Each 3 cm umbilical cord segment was dissected longitudinally utilizing aseptic technique. The tissue was carefully undermined and the umbilical vein and both umbilical arteries were removed. The remaining segment was sutured inside out and incubated in 25 ml of PBS, 1.times. antibiotic, and 1 mg/ml of collagenase at room temperature. After 16-18 hours the remaining suture and connective tissue was removed and discarded. The cell suspension was separated equally into two tubes, the cells were washed 3.times. by diluting with PBS to yield a final volume of 50 ml per tube, and then centrifuged. Red blood cells were then lysed using a hypotonic solution. Cells were plated onto 6-well plates at a concentration of 5-20.times.10.sup.6 cells per well. UC-MSC were cultured in low-glucose DMEM (Gibco) with 10% FBS (Hyclone), 2 mM L-Glutamine (Gibco), 100 U/ml penicillin, 100 ug/ml streptomycin, 0.025 ug/ml amphotericin B (Gibco). Cells were washed 48 hours after the initial plating with PBS and given fresh media. Cell culture media were subsequently changed twice a week through half media changes. After 7 days or approximately 70-80% confluence, cells were passed using HyQTase (Hyclone) into a 10 cm plate. Cells were then regularly passed 1:2 every 7 days or upon reaching 80% confluence. Alternatively, 0.25% HQ trypsin/EDTA (Hyclone) was used to passage cells in a similar manner.

In some embodiments of the invention, administration of cells of the invention is performed for suppression of an inflammatory and/or autoimmune disease. In these situations, it may be necessary to utilize an immune suppressive/or therapeutic adjuvant. Immune suppressants are known in the art and can be selected from a group comprising of: cyclosporine, rapamycin, campath-1H, ATG, Prograf, anti IL-2r, MMF, FTY, LEA, cyclosporin A, diftitox, denileukin, levamisole, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, and trimetrexate), purine analogs (e.g., cladribine, fludarabine, 6-mercaptopurine, thiamiprine, and thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, and tegafur) fluocinolone, triaminolone, anecortave acetate, fluorometholone, medrysone, prednislone, etc. In another embodiment, the use of stem cell conditioned media may be used to potentiate an existing anti-inflammatory agent. Anti-inflammatory agents may comprise one or more agents including NSAIDs, interleukin-1 antagonists, dihydroorotate synthase inhibitors, p38 MAP kinase inhibitors, TNF-α inhibitors, TNF-α sequestration agents, and methotrexate. More specifically, anti-inflammatory agents may comprise one or more of, e.g., anti-TNF-α, lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (eg., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric.acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid.

The introduction of “biological therapies” such as anti-TNF-alpha antibodies has led to some improvements in prognosis, although side effects are still present due to the non-specific nature of the intervention. The same holds true for cytokine storm conditions such as sepsis, where overproduction of agents such as TNF-alpha result in vascular leakage, coagulopathy, and death. The invention provides the utilization of tolerance-induction using regenerative cells, in one embodiment, said regenerative cells are capable of inducing infectious tolerance alone, or in combination with existing techniques. The utilization of antigen-nonspecific immature dendritic cells which are generated by co-culture with regenerative cells allows for induction of a inhibitory immune response, which results in suppression of autoimmune or alloimmune inflammation. The invention teaches that to effectively treat conditions of immune overactivation it is important to delete/inactivate the T cell clone that are associated with stimulation of inflammation, as well as to block innate immune elements. This would be akin to recapitulating the natural process of tolerance induction. While thymic deletion was the original process identified as being responsible for selectively deleting autoreactive T cells, it became clear that numerous redundant mechanisms exist that are not limited to the neonatal period. Specifically, a “mirror image” immune system was demonstrated to co-exist with the conventional immune system. Conventional T cells are activated by self-antigens to die in the thymus and conventional T cells that are not activated receive a survival signal [1]; the “mirror image”, T regulatory (Treg) cells are actually selected to live by encounter with self-antigens, and Treg cells that do not bind self antigens are deleted [2, 3]. In one embodiment of the invention, immature dendritic cells are administered in order to induce a state of immune modulation, including T regulatory cell generation by the immature dendritic cells. Utilization of immature dendritic cells to stimulate T regulatory cell proliferation and/or activity has been previously demonstrated and is incorporated by reference [4-10].

Thus the self-nonself discrimination by the immune system occurs in part based on self antigens depleting autoreactive T cells, while promoting the generation of Treg cells. An important point for development of an antigen-specific tolerogenic vaccine is that in adult life, and in the periphery, autoreactive T cells are “energized” by presentation of self-antigens in absence of danger signals, and autoreactive Treg are generated in response to self antigens. Although the process of T cell deletion in the thymus is different than induction of T cell anergy, and Treg generation in the thymus, results in a different type of Treg as compared to peripheral induced Treg, in many aspects, the end result of adult tolerogenesis is similar to that which occurs in the neonatal period.

Specific examples of tolerogenesis that occurs in adults includes settings such as pregnancy, cancer, and oral tolerance. In the situation of pregnancy, studies have demonstrated selective inactivation of maternal T cell clones that recognize fetal antigens occurs through a variety of mechanisms, including FasL expression on fetal and placental cells [11], antigen presentation in the context of PD1-L [12], and HLA-G interacting with immune inhibitory receptors such as ILT4 [13]. In pregnancy, “tolerogenic antigen presentation” occurs only through the indirect pathway of antigen presentation [14]. Other pathways of selective tolerogenesis in pregnancy include the stimulation of Treg cells, which have been demonstrated essential for successful pregnancy [15]. In the context of cancer, depletion of tumor specific T cells, while sparing of T cells with specificities to other antigens has been demonstrated by the tumor itself or tumor associated cells [16-19]. Additionally, Treg cells have been demonstrated to actively suppress anti-tumor T cells, perhaps as a “back up” mechanism of tumor immune evasion [20-22]. At a clinical level the ability of tumors to inhibit peripheral T cell activity has been associated in numerous studies with poor prognosis [23-25]. Oral tolerance is the process by which ingested antigens induce generation of antigen-specific TGF-beta producing cells (called “Th3” by some) [26-28], as well as Treg cells [29, 30]. Ingestion of antigen, including the autoantigen collagen II [31], has been shown to induce inhibition of both T and B cell responses in a specific manner [32, 33]. It appears that induction of regulatory cells, as well as deletion/anergy of effector cells is associated with antigen presentation in a tolerogenic manner [34]. Remission of disease in animal models of RA [35], multiple sclerosis [36], and type I diabetes [37], has been reported by oral administration of autoantigens. Furthermore, clinical trials have shown signals of efficacy of oral tolerance in autoimmune diseases such as rheumatoid arthritis [38], autoimmune uveitis [39], and multiple sclerosis [40]. In all of these natural conditions of tolerance, common molecules and mechanisms seem to be operating. Accordingly, a natural means of inducing tolerance would be the administration of a “universal donor” cell with tolerogenic potential that generate molecules similar to those found in physiological conditions of tolerance induction.

In some embodiments of the invention the generation of immature dendritic cells by culture with regenerative cells such as amniotic fluid stem cells is performed by either coculture in vitro, or administration in vivo of T regulatory cells [41].

In some embodiments of the invention, alpha 1 antitrypsin is administered in order to induce tolerogenic dendritic cells in order to treat autoimmunity or alloimmunity. The use of this compound for stimulation of immature DC has been previously described and is incorporated by reference [42].

In one embodiment, the invention teaches reduction of Inflammatory cytokines, especially tumor necrosis factor alpha (TNF) and interleukin 1-beta (IL-1), by administration of patient immune cells treated ex vivo with regenerative cells such as amniotic fluid stem cells, wherein in some embodiments said amniotic fluid stem cells are also cultured with immature dendritic cells. It is known that these inflammatory cytokines are major mediators that can elicit changes in cell phenotype, especially causing a variety of morphological and gene expression changes in endothelial cells. In one embodiment treatment of blood vessel abnormalities such as hypercoagulability is treated using patient cells that have been reprogrammed by amniotic fluid stem cells.

In some embodiments of the invention, reprogrammed immune cells are administered together with drugs useful for treatment of inflammatory conditions such as Xigris (activated protein C (APC)) [43]. This protein we claim synergizes with the anti-inflammatory effects of patient reprogrammed immune cells by activating endothelial cell-protecting mechanisms mediating protection against apoptosis, stimulation of barrier function through the angiopoietin/Tie-2 axis, and by reducing local clotting [44-46]. The basis of approval for Xigris has been questioned by some [47] and, additionally, it is often counter-indicated in oncology-associated sepsis (especially leukemias where bleeding is an issue of great concern). In fact, in the Phase III trials of Xigris, hematopoietic transplant patients were excluded [48]. Thus there is a great need for progress in the area of SIRS treatment and adjuvant approaches for agents such as Xigris. In one embodiment of the invention, APC is administered as Xigris.

In one embodiment, cells of the invention are used to treated systemic inflammatory response syndrome (SIRS). One of the main causes of death related to SIRS is dysfunction of the microcirculatory system, which in the most advanced stages is manifested as disseminated intravascular coagulation (DIC) [49]. In one embodiment, patient immune cells that have been reprogrammed with amniotic fluid stem cells are administered together with immature dendritic cells to inhibit onset of DIC. Without being bound to theory, immature dendritic cells are generated in a manner to inhibit inflammatory mediators associated with SIRS, whether endotoxin or injury-related signals such as TLR agonists or HMGB-1, are all capable of activating endothelium systemically [50, 51]. Under physiological conditions, the endothelial response to such mediators is local and provides a useful mechanism for sequestering an infection and allowing immune attack. In SIRS, the fact that the response is systemic causes disastrous consequences including organ failure. The characteristics of this endothelial response include: a) upregulation of tissue factor (TF) [52, 53] and suppression of endothelial inhibitors of coagulation such as protein C and the antithrombin system causing a pro-coagulant state [54]; b) increased expression of adhesion molecules which elicit, in turn, neutrophil extravasation [55]; c) decreased fibrinolytic capacity [56-58]; and d) increased vascular permeability/non-responsiveness to vaso-dilators and vasoconstrictors [59, 60]. Excellent detailed reviews of molecular signals associated with SIRS-induced endothelial dysfunction have been published[61-69] and one of the key factors implicated has been NF-kB [70]. Nuclear translocation of NF-kB is associated with endothelial upregulation of pro-thrombotic molecules and suppressed fibrinolysis [71-73]. In an elegant study, Song et al. inhibited NF-kB selectively in the endothelium by creation of transgenic mice transgenic expressing exogenous i-kappa B (the NF-kB inhibitor) specifically in the vasculature. In contrast to wild-type animals, the endothelial cells of these transgenic mice experienced substantially reduced expression of tissue factor while retaining expression of endothelial protein C receptor and thrombomodulin subsequent to endotoxin challenge. Furthermore, expression of NF-B was associated with generation of TNF-alpha as a result of TACE activity [74]. It is interesting that the beneficial effects of Xigris in SIRS appear to be associated with its ability to prevent the endothelial dysfunction [75] associated with suppression of proinflammatory chemokines [76], prevention of endothelial cell apoptosis [77], and increased endothelial fibrinolytic activity [78, 79]. Some of the protective activities of Xigris have been ascribed to its ability to suppress NF-kB activation in endothelial cells [80, 81].

### Example 1: Resolution of Rheumatoid Arthritis by Administration of Amniotic Stem Cell Conditioned Splenocytes

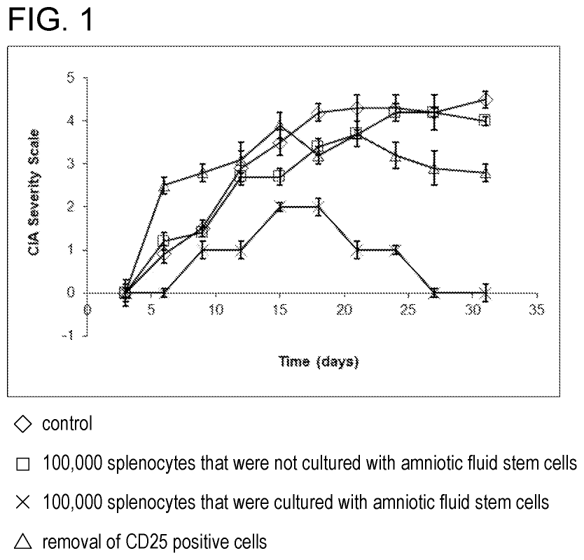

Collagen induced arthritis was elicited in mice as previously described [82], 12 mice where used per group. Murine splenocytes were co-cultured with amniotic fluid stem cells for 3 days in the RPMI complete media and subsequently T cells were purified by negative depletion and added to mice 14 days after last collagen injection. As seen in FIG. 1, control (diamond) mice had rapid onset of disease, as well as mice injected with 100,000 splenocytes that were not cultured with amniotic fluid stem cells (square). Administration of 100,000 splenocytes that were cultured with amniotic fluid stem cells (X) resulted in disease protection. Depletion of T regulatory cells by removal of CD25 positive cells resulted in negation of protection (triangle). These data suggest that administration of immune cells subsequent to coculture with amniotic fluid stem cells results in T regulatory cell dependent protection. Interestingly, transfer of 100,000 splenocytes from protected mice to naïve mice endows naïve mice with protection from rheumatoid arthritis.

### Example 2: Administration of Amniotic Stem Cell Conditioned Splenocytes Increases Tolerogenic Dendritic Cells In Vivo

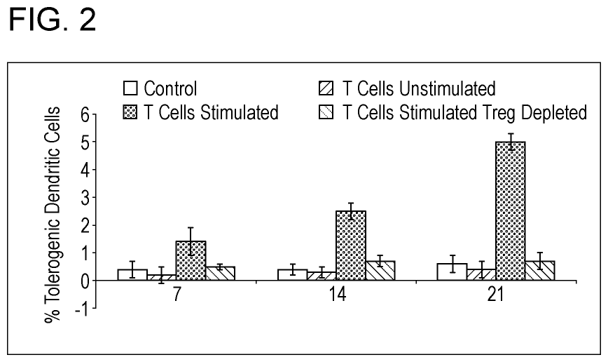

Collagen induced arthritis was elicited in mice as previously described [82], 12 mice where used per group. Murine splenocytes were co-cultured with amniotic fluid stem cells for 3 days in the RPMI complete media and subsequently T cells were purified by negative depletion and added to mice 14 days after last collagen injection. As shown in FIG. 2, control mice had little numbers of tolerogenic dendritic cells (assessed by CD11c and IL-10 staining, as well as mice injected with 100,000 splenocytes that were not cultured with amniotic fluid stem cells. Administration of 100,000 splenocytes that were cultured with amniotic fluid stem cells resulted in augmented numbers of tolerogenic DC. Depletion of T regulatory cells by removal of CD25 positive cells resulted in tolerogenic dendritic cell increase. These data suggest that administration of immune cells subsequent to coculture with amniotic fluid stem cells results in T regulatory cell dependent generation of tolerogenic dendritic cells. Animals were sacrificed on days 7, 14 and 21.

### REFERENCES

1. Ramsdell, F. and B. J. Fowlkes, Clonal deletion versus clonal anergy: the role of the thymus in inducing self tolerance. Science, 1990. 248(4961): p. 1342-8.
2. Apostolou, I., et al., Origin of regulatory T cells with known specificity for antigen. Nat Immunol, 2002. 3(8): p. 756-63.
3. Aschenbrenner, K., et al., Selection of Foxp3+ regulatory T cells specific for self antigen expressed and presented by Aire+ medullary thymic epithelial cells. Nat Immunol, 2007. 8(4): p. 351-8.
4. Cong, Y., et al., Generation of antigen-specific, Foxp3-expressing CD4+ regulatory T cells by inhibition of APC proteosome function. J Immunol, 2005. 174(5): p. 2787-95.
5. Buckland, M., et al., Aspirin-treated human DCs up-regulate ILT-3 and induce hyporesponsiveness and regulatory activity in responder T cells. Am J Transplant, 2006. 6(9): p. 2046-59.
6. Jin, Y., et al., Induction of auto-reactive regulatory T cells by stimulation with immature autologous dendritic cells. Immunol Invest, 2007. 36(2): p. 213-32.
7. Gandhi, R., D. E. Anderson, and H. L. Weiner, Cutting Edge: Immature human dendritic cells express latency-associated peptide and inhibit T cell activation in a TGF-beta-dependent manner. J Immunol, 2007. 178(7): p. 4017-21.
8. Gaudreau, S., et al., Granulocyte-macrophage colony-stimulating factor prevents diabetes development in NOD mice by inducing tolerogenic dendritic cells that sustain the suppressive function of CD4+CD25+ regulatory T cells. J Immunol, 2007. 179(6): p. 3638-47.
9. Zhang, X., et al., Generation of therapeutic dendritic cells and regulatory T cells for preventing allogeneic cardiac graft rejection. Clin Immunol, 2008. 127(3): p. 313-21.
10. Marguti, I., et al., Expansion of CD4+ CD25+ Foxp3+ T cells by bone marrow-derived dendritic cells. Immunology, 2009. 127(1): p. 50-61.
11. Vacchio, M. S. and R. J. Hodes, Fetal expression of Fas ligand is necessary and sufficient for induction of CD8 T cell tolerance to the fetal antigen H-Y during pregnancy. J Immunol, 2005. 174(8): p. 4657-61.
12. D'Addio, F., et al., The link between the PDL1 costimulatory pathway and Th17 in fetomaternal tolerance. J Immunol, 2011. 187(9): p. 4530-41.
13. Kuroki, K. and K. Maenaka, Immune modulation of HLA-G dimer in maternal-fetal interface. Eur J Immunol, 2007. 37(7): p. 1727-9.
14. Erlebacher, A., et al., Constraints in antigen presentation severely restrict T cell recognition of the allogeneic fetus. J Clin Invest, 2007. 117(5): p. 1399-411.
15. Chen, T., et al., Self-specific memory regulatory T cells protect embryos at implantation in mice. J Immunol, 2013. 191(5): p. 2273-81.
16. Harimoto, H., et al., Inactivation of tumor-specific CD8(+) CTLs by tumor-infiltrating tolerogenic dendritic cells. Immunol Cell Biol, 2013. 91(9): p. 545-55.
17. Ney, J. T., et al., Autochthonous liver tumors induce systemic T cell tolerance associated with T cell receptor down-modulation. Hepatology, 2009. 49(2): p. 471-81.
18. Cheung, A. F., et al., Regulated expression of a tumor-associated antigen reveals multiple levels of T-cell tolerance in a mouse model of lung cancer. Cancer Res, 2008. 68(22): p. 9459-68.
19. Bal, A., et al., Rapid tolerization of virus-activated tumor-specific CD8+ T cells in prostate tumors of TRAMP mice. Proc Natl Acad Sci USA, 2008. 105(35): p. 13003-8.
20. Jacobs, J. F., et al., Regulatory T cells in melanoma: the final hurdle towards effective immunotherapy? Lancet Oncol, 2012. 13(1): p. e32-42.
21. Pedroza-Gonzalez, A., et al., Activated tumor-infiltrating CD4+ regulatory T cells restrain antitumor immunity in patients with primary or metastatic liver cancer. Hepatology, 2013. 57(1): p. 183-94.
22. Donkor, M. K., et al., T cell surveillance of oncogene-induced prostate cancer is impeded by T cell-derived TGF-beta1 cytokine. Immunity, 2011. 35(1): p. 123-34.
23. Whiteside, T. L., Down-regulation of zeta-chain expression in T cells: a biomarker of prognosis in cancer? Cancer Immunol Immunother, 2004. 53(10): p. 865-78.
24. Whiteside, T. L., Signaling defects in T lymphocytes of patients with malignancy. Cancer Immunol Immunother, 1999. 48(7): p. 346-52.
25. Reichert, T. E., et al., Signaling abnormalities, apoptosis, and reduced proliferation of circulating and tumor-infiltrating lymphocytes in patients with oral carcinoma. Clin Cancer Res, 2002. 8(10): p. 3137-45.
26. Faria, A. M. and H. L. Weiner, Oral tolerance. Immunol Rev, 2005. 206: p. 232-59.
27. Weiner, H. L., Induction and mechanism of action of transforming growth factor-beta-secreting Th3 regulatory cells. Immunol Rev, 2001. 182: p. 207-14.
28. Fukaura, H., et al., Induction of circulating myelin basic protein and proteolipid protein-specific transforming growth factor-beta1-secreting Th3 T cells by oral administration of myelin in multiple sclerosis patients. J Clin Invest, 1996. 98(1): p. 70-7.
29. Palomares, O., et al., Induction and maintenance of allergen-specific FOXP3+ Treg cells in human tonsils as potential first-line organs of oral tolerance. J Allergy Clin Immunol, 2012. 129(2): p. 510-20, 520 el-9.
30. Yamashita, H., et al., Overcoming food allergy through acquired tolerance conferred by transfer of Tregs in a murine model. Allergy, 2012. 67(2): p. 201-9.
31. Park, K. S., et al., Type II collagen oral tolerance; mechanism and role in collagen-induced arthritis and rheumatoid arthritis. Mod Rheumatol, 2009.
32. Womer, K. L., et al., A pilot study on the immunological effects of oral administration of donor major histocompatibility complex class II peptides in renal transplant recipients. Clin Transplant, 2008. 22(6): p. 754-9.
33. Faria, A. M. and H. L. Weiner, Oral tolerance: therapeutic implications for autoimmune diseases. Clin Dev Immunol, 2006. 13(2-4): p. 143-57.
34. Park, M. J., et al., A distinct tolerogenic subset of splenic IDO(+)CD11b(+) dendritic cells from orally tolerized mice is responsible for induction of systemic immune tolerance and suppression of collagen-induced arthritis. Cell Immunol, 2012. 278(1-2): p. 45-54.
35. Thompson, H. S., et al., Suppression of collagen induced arthritis by oral administration of type II collagen: changes in immune and arthritic responses mediated by active peripheral suppression. Autoimmunity, 1993. 16(3): p. 189-99.
36. Song, F., et al., The thymus plays a role in oral tolerance induction in experimental autoimmune encephalomyelitis. Ann N Y Acad Sci, 2004. 1029: p. 402-4.
37. Hanninen, A. and L. C. Harrison, Mucosal tolerance to prevent type 1 diabetes: can the outcome be improved in humans? Rev Diabet Stud, 2004. 1(3): p. 113-21.
38. Wei, W., et al., A multicenter, double-blind, randomized, controlled phase III clinical trial of chicken type II collagen in rheumatoid arthritis. Arthritis Res Ther, 2009. 11(6): p. R180.
39. Thurau, S. R., et al., Molecular mimicry as a therapeutic approach for an autoimmune disease: oral treatment of uveitis-patients with an MHC peptide crossreactive with autoantigen—first results. Immunol Lett, 1997. 57(1-3): p. 193-201.
40. Weiner, H. L., et al., Double-blind pilot trial of oral tolerization with myelin antigens in multiple sclerosis. Science, 1993. 259(5099): p. 1321-4.
41. Onishi, Y., et al., Foxp3+ natural regulatory T cells preferentially form aggregates on dendritic cells in vitro and actively inhibit their maturation. Proc Natl Acad Sci USA, 2008. 105(29): p. 10113-8.
42. Ozeri, E., et al., alpha-1 antitrypsin promotes semimature, IL-10-producing and readily migrating tolerogenic dendritic cells. J Immunol, 2012. 189(1): p. 146-53.
43. Ely, E. W., G. R. Bernard, and J. L. Vincent, Activated protein C for severe sepsis. N Engl J Med, 2002. 347(13): p. 1035-6.
44. Dhainaut, J. F., et al., Drotrecogin alfa (activated) (recombinant human activated protein C) reduces host coagulopathy response in patients with severe sepsis. Thromb Haemost, 2003. 90(4): p. 642-53.
45. Minhas, N., et al., Activated protein C utilizes the angiopoietin/Tie2 axis to promote endothelial barrier function. FASEB J. 24(3): p. 873-81.
46. Loubele, S. T., H. M. Spronk, and H. Ten Cate, Activated protein C: a promising drug with multiple effects? Mini Rev Med Chem, 2009. 9(5): p. 620-6.
47. Poole, D., G. Bertolini, and S. Garattini, Errors in the approval process and post-marketing evaluation of drotrecogin alfa (activated) for the treatment of severe sepsis. Lancet Infect Dis, 2009. 9(1): p. 67-72.
48. Pastores, S. M., et al., Septic shock and multiple organ failure after hematopoietic stem cell transplantation: treatment with recombinant human activated protein C. Bone Marrow Transplant, 2002. 30(2): p. 131-4.
49. Gando, S., Disseminated intravascular coagulation in trauma patients. Semin Thromb Hemost, 2001. 27(6): p. 585-92.
50. Cristofaro, P. and S. M. Opal, The Toll-like receptors and their role in septic shock. Expert Opin Ther Targets, 2003. 7(5): p. 603-12.
51. Treutiger, C. J., et al., High mobility group 1 B-box mediates activation of human endothelium. J Intern Med, 2003. 254(4): p. 375-85.
52. Lv, B., et al., High-mobility group box 1 protein induces tissue factor expression in vascular endothelial cells via activation of NF-kappaB and Egr-1. Thromb Haemost, 2009. 102(2): p. 352-9.
53. Wada, H., Y. Wakita, and H. Shiku, Tissue factor expression in endothelial cells in health and disease. Blood Coagul Fibrinolysis, 1995. 6 Suppl 1: p. S26-31.
54. Levi, M., The coagulant response in sepsis and inflammation. Hamostaseologie. 30(1): p. 10-2, 14-6.
55. Munro, J. M., J. S. Pober, and R. S. Cotran, Recruitment of neutrophils in the local endotoxin response: association with de novo endothelial expression of endothelial leukocyte adhesion molecule-1. Lab Invest, 1991. 64(2): p. 295-9.
56. Lippi, G., L. Ippolito, and G. Cervellin, Disseminated intravascular coagulation in burn injury. Semin Thromb Hemost. 36(4): p. 429-36.
57. Lau, C. L., et al., Enhanced fibrinolysis protects against lung ischemia-reperfusion injury. J Thorac Cardiovasc Surg, 2009. 137(5): p. 1241-8.
58. Levi, M., M. Schouten, and T. van der Poll, Sepsis, coagulation, and antithrombin: old lessons and new insights. Semin Thromb Hemost, 2008. 34(8): p. 742-6.
59. Shapiro, N., et al., The association of endothelial cell signaling, severity of illness, and organ dysfunction in sepsis. Crit Care. 14(5): p. R182.
60. Druey, K. M. and P. R. Greipp, Narrative review: the systemic capillary leak syndrome. Ann Intern Med. 153(2): p. 90-8.
61. Dejana, E., F. Orsenigo, and M. G. Lampugnani, The role of adherens junctions and VE-cadherin in the control of vascular permeability. J Cell Sci, 2008. 121(Pt 13): p. 2115-22.
62. Azevedo, L. C., et al., Redox mechanisms of vascular cell dysfunction in sepsis. Endocr Metab Immune Disord Drug Targets, 2006. 6(2): p. 159-64.
63. Okajima, K., Prevention of endothelial cell injury by activated protein C: the molecular mechanism(s) and therapeutic implications. Curr Vasc Pharmacol, 2004. 2(2): p. 125-33.
64. Andersson, U. and K. J. Tracey, HMGB1 in sepsis. Scand J Infect Dis, 2003. 35(9): p. 577-84.
65. Strassheim, D., J. S. Park, and E. Abraham, Sepsis: current concepts in intracellular signaling. Int J Biochem Cell Biol, 2002. 34(12): p. 1527-33.
66. ten Cate, H., et al., Microvascular coagulopathy and disseminated intravascular coagulation. Crit Care Med, 2001. 29(7 Suppl): p. S95-7; discussion S97-8.
67. Hawiger, J., Innate immunity and inflammation: a transcriptional paradigm. Immunol Res, 2001. 23(2-3): p. 99-109.
68. Edgington, T. S., et al., Cellular immune and cytokine pathways resulting in tissue factor expression and relevance to septic shock. Nouv Rev Fr Hematol, 1992. 34 Suppl: p. S15-27.
69. Mukaida, N., et al., Novel insight into molecular mechanism of endotoxin shock: biochemical analysis of LPS receptor signaling in a cell-free system targeting NF-kappaB and regulation of cytokine production/action through beta2 integrin in vivo. J Leukoc Biol, 1996. 59(2): p. 145-51.
70. Liu, S. F. and A. B. Malik, NF-kappa B activation as a pathological mechanism of septic shock and inflammation. Am J Physiol Lung Cell Mol Physiol, 2006. 290(4): p. L622-L645.
71. Ulfhammer, E., et al., TNF-alpha mediated suppression of tissue type plasminogen activator expression in vascular endothelial cells is NF-kappaB- and p38 MAPK-dependent. J Thromb Haemost, 2006. 4(8): p. 1781-9.
72. Xu, H., et al., Selective blockade of endothelial NF-kappaB pathway differentially affects systemic inflammation and multiple organ dysfunction and injury in septic mice. J Pathol. 220(4): p. 490-8.
73. Ding, J., et al., A pivotal role of endothelial-specific NF-kappaB signaling in the pathogenesis of septic shock and septic vascular dysfunction. J Immunol, 2009. 183(6): p. 4031-8.
74. Song, D., et al., Activation of endothelial intrinsic NF-{kappa}B pathway impairs protein C anticoagulation mechanism and promotes coagulation in endotoxemic mice. Blood, 2009. 114(12): p. 2521-9.
75. Grinnell, B. W. and D. Joyce, Recombinant human activated protein C: a system modulator of vascular function for treatment of severe sepsis. Crit Care Med, 2001. 29(7 Suppl): p. S53-60; discussion S60-1.
76. Brueckmann, M., et al., Activated protein C inhibits the release of macrophage inflammatory protein-1-alpha from THP-1 cells and from human monocytes. Cytokine, 2004. 26(3): p. 106-13.
77. Cheng, T., et al., Activated protein C blocks p53-mediated apoptosis in ischemic human brain endothelium and is neuroprotective. Nat Med, 2003. 9(3): p. 338-42.
78. van Hinsbergh, V. W., et al., Activated protein C decreases plasminogen activator-inhibitor activity in endothelial cell-conditioned medium. Blood, 1985. 65(2): p. 444-51.
79. Sakata, Y., et al., Activated protein C stimulates the fibrinolytic activity of cultured endothelial cells and decreases antiactivator activity. Proc Natl Acad Sci USA, 1985. 82(4): p. 1121-5.
80. Joyce, D. E. and B. W. Grinnell, Recombinant human activated protein C attenuates the inflammatory response in endothelium and monocytes by modulating nuclear factor-kappaB. Crit Care Med, 2002. 30(5 Suppl): p. S288-93.
81. Brueckmann, M., et al., Drotrecogin alfa (activated) inhibits NF-kappa B activation and MIP-1-alpha release from isolated mononuclear cells of patients with severe sepsis. Inflamm Res, 2004. 53(10): p. 528-33.
82. Popov, I., et al., Preventing autoimmune arthritis using antigen-specific immature dendritic cells: a novel tolerogenic vaccine. Arthritis Res Ther, 2006. 8(5): p. R141.

1. A method of treating autoimmunity or alloreactivity in a patient comprising the steps of: a) obtaining immune cells from a patient suffering from autoimmunity or alloimmunity; b) contacting said immune cells with a regenerative cell; c) providing an antigen presenting cell in the mixture in a manner in which said antigen presenting cell is capable of generating a tolerogenic reaction with said immune cells from said patient suffering from autoimmunity or alloimmunityl and d) administering said immune cells back into said patient.

2. The method of claim 1, wherein said immune cells are selected from a group of cells comprising of: a) peripheral blood mononuclear cells; b) T cells; c) B cells; d) ILC; e) gamma delta T cells; f) nk cells; g) NKT cells; h) monocytes; i) dendritic cells; and j) neutrophils.

3. The method of claim 1, wherein said antigen presenting cell is a myeloid cell.

4. The method of claim 3, wherein said myeloid cell is a dendritic cell.

5. The method of claim 4, wherein said dendritic cell is tolerogenic.

6. The method of claim 5, wherein said tolerogenic dendritic cell is immature.

7. The method of claim 6, wherein said immature dendritic cell is made immature by culture with an inhibitor of NF-kappa B.

8. The method of claim 7, wherein said inhibitor of NF-kappa B is interleukin-10.

9. The method of claim 7, wherein said inhibitor of NF-kappa B is VEGF.

10. The method of claim 7, wherein said inhibitor of NF-kappa B is IGF-1

11. The method of claim 7, wherein said inhibitor of NF-kappa B is HGF-1.

12. The method of claim 7, wherein said inhibitor of NF-kappa B is BDNF.

13. The method of claim 7, wherein said inhibitor of NF-kappa B is hCG.

14. The method of claim 1, wherein said contact of said regenerative cell with said immune cell is performed by transfer of conditioned media.

15. The method of claim 14, wherein said conditioned media is generated by treating said mesenchymal stem cells under hypoxia.

16. The method of claim 14, wherein said conditioned media is generated by treating said mesenchymal stem cells under inflammatory stress.

17. The method of claim 16, wherein said inflammatory stress is oxidative stress.

18. The method of claim 17, wherein said oxidative stress is treatment with ozone gas at a concentration sufficient to stimulate an increase in SOD1 transcription of more than 25% as compared to baseline.

19. The method of claim 1, wherein said regenerative cell is a mesenchymal stem cell.

20. The method of claim 19, wherein said mesenchymal stem cell is an umbilical cord mesenchymal stem cell.
